SEQUENCE LISTING (25 sequences)

SEQ ID NO: 1 - protein, 10 aa, synthetic construct
VDFEAFWDWG

SEQ ID NO: 2 - DNA, 20 bp, synthetic construct
  source 1..20: mol_type = other DNA
tgctggtgctgagtatgtcg

SEQ ID NO: 3 - DNA, 20 bp, synthetic construct
  source 1..20: mol_type = other DNA
caagcagttggtggtacagg

SEQ ID NO: 4 - DNA, 20 bp, synthetic construct
  source 1..20: mol_type = other DNA
acgctaccacggaaacaatc

SEQ ID NO: 5 - DNA, 20 bp, synthetic construct
  source 1..20: mol_type = other DNA
ggagtcttgacgggtctgag

SEQ ID NO: 6 - DNA, 20 bp, synthetic construct
  source 1..20: mol_type = other DNA
tccagtacattgagcgccta

SEQ ID NO: 7 - DNA, 20 bp, synthetic construct
  source 1..20: mol_type = other DNA
caaatgatctcctgggttgg

SEQ ID NO: 8 - DNA, 20 bp, synthetic construct
  source 1..20: mol_type = other DNA
aaagaggctccaggtccaat

SEQ ID NO: 9 - DNA, 20 bp, synthetic construct
  source 1..20: mol_type = other DNA
cctctctgcttgtgtggtca

SEQ ID NO: 10 - DNA, 20 bp, synthetic construct
  source 1..20: mol_type = other DNA
gggttccattgacattgacc

SEQ ID NO: 11 - DNA, 20 bp, synthetic construct
  source 1..20: mol_type = other DNA
agggccagtgtttcacattc

SEQ ID NO: 12 - DNA, 20 bp, synthetic construct
  source 1..20: mol_type = other DNA
ttcagcagcctgaactacga

SEQ ID NO: 13 - DNA, 20 bp, synthetic construct
  source 1..20: mol_type = other DNA
tgaaagcttcccccaaagta

SEQ ID NO: 14 - DNA, 20 bp, synthetic construct
  source 1..20: mol_type = other DNA
tgaggtgcctacttgctcct

SEQ ID NO: 15 - DNA, 20 bp, synthetic construct
  source 1..20: mol_type = other DNA
tcacctggtggctattctcc

SEQ ID NO: 16 - DNA, 20 bp, synthetic construct
  source 1..20: mol_type = other DNA
aacttccagagagacgcctt

SEQ ID NO: 17 - DNA, 20 bp, synthetic construct
  source 1..20: mol_type = other DNA
atcgtgggcctcatcttctt

SEQ ID NO: 18 - DNA, 20 bp, synthetic construct
  source 1..20: mol_type = other DNA
tgcagaagatccctcctgtc

SEQ ID NO: 19 - DNA, 20 bp, synthetic construct
  source 1..20: mol_type = other DNA
cttgatctcgttcccatcca

SEQ ID NO: 20 - DNA, 20 bp, synthetic construct
  source 1..20: mol_type = other DNA
tggagctgttattggtgcag

SEQ ID NO: 21 - DNA, 20 bp, synthetic construct
  source 1..20: mol_type = other DNA
tgggttttgcacatcaaaga

SEQ ID NO: 22 - DNA, 20 bp, synthetic construct
  source 1..20: mol_type = other DNA
aagagctgacccaatggttg

SEQ ID NO: 23 - DNA, 20 bp, synthetic construct
  source 1..20: mol_type = other DNA
acccttgcatccttcacaag

SEQ ID NO: 24 - DNA, 20 bp, synthetic construct
  source 1..20: mol_type = other DNA
ctggtcgtgtggaagtgaaa

SEQ ID NO: 25 - DNA, 20 bp, synthetic construct
  source 1..20: mol_type = other DNA
cgccactgagcatagtgaaa

(19) United States
(12) Patent Application Publication
(10) Pub. No.: US 2025/0090626 A1
(43) Pub. Date: Mar. 20, 2025

(54) NEW PEPTIDE AND USES THEREOF

(71) Applicant: Neo Cremar Co., Ltd., Seoul (KR)

(72) Inventors: Inho CHOI, Gyeongsan-si, Gyeongsangbuk-do (KR); Eun Ju LEE, Daegu (KR); So-young PARK, Daegu (KR); Mohammad Hassan BAIG, Incheon (KR); Khurshid AHMAD, Gyeongsan-si, Gyeongsangbuk-do (KR); Sibhghatulla SHAIKH, Gyeongsan-si, Gyeongsangbuk-do (KR); Syed Sayeed AHMAD, Gyeongsan-si, Gyeongsangbuk-do (KR)

(73) Assignee: Neo Cremar Co., Ltd., Seoul (KR)

(21) Appl. No.: 18/712,697

(22) PCT Filed: Nov. 11, 2022
(86) PCT No.: PCT/KR2022/017769

§ 371 Date: May 22, 2024

(30) Foreign Application Priority Data
Nov. 25, 2021 (KR) 10-2021-0164344
Nov. 25, 2021 (KR) 10-2021-0164345
May 11, 2022 (KR) 10-2022-0057546
May 11, 2022 (KR) 10-2022-0057547

(51) Int. Cl. A61K 38/16; C07K 14/00; C12N 5/077

(52) U.S. Cl. CPC A61K 38/16; C07K 14/001; C12N 5/0658; C12N 2501/998

(57) ABSTRACT

The present invention relates to a novel peptide for promoting the proliferation and differentiation of myoblasts or inhibiting the proliferation and differentiation of preadipocytes, and use thereof. A novel peptide is designed on the basis of an amino acid site, from among parts of a myostatin (MSTN) protein, binding to a fibromodulin protein, and then: C2C12 cells, which are a mouse myoblast cell line, are treated with the peptide so that cell proliferation and differentiation, muscle regeneration and the like are observed, and it has been identified that the proliferation and differentiation of myoblasts and the regeneration of muscles increase after the treatment with the designed peptide; and 3T3L1 cells, which are mouse preadipocytes, are treated with the peptide so that cell proliferation, fat differentiation, and the like are observed, and it has been identified that the proliferation of preadipocytes and fat differentiation decrease after the treatment with the peptide.

### TECHNICAL FIELD

The present disclosure relates to a novel peptide and a use thereof, and specifically, provides a novel peptide, which promotes proliferation of myoblasts and differentiation into myocytes or suppresses proliferation of preadipocytes and differentiation into adipocytes, and a use thereof.

### BACKGROUND ART

Muscle is an important element of the human body and a tissue expressed from stem cells of mesoderm. Taking up about 40% of our body, muscles are positioned in support of bones and tendons and made up of bundles of myofibers to move together, inducing contraction by varying the size of cells. Muscles are divided into skeletal muscles, heart muscles, and visceral muscles, each of which generates force and causes movement from its position, while playing a role in protecting body organs such as bones, joints, and internal organs. In addition, muscles have an ability to regenerate, so when a muscle is damaged, it may be degenerated by satellite cells and its surrounding environment and then regenerated into a muscle with its original contraction and relaxation abilities.

Muscle diseases are caused by congenital genetics or environmental causes, and in recent years, diseases related to muscle loss have been increasing with the aging society and life extension. Since the human muscles dwindle down by more than 1% every year after the age of 40, and the maximum muscle mass level is reduced by 50% by the age of 80, the muscle loss in old age is recognized as the most important cause of deterioration in overall physical functions. These muscle diseases are on the rise worldwide compared to the past.

However, it is not easy to accurately diagnose muscle diseases because their causes are more diverse than other diseases, and the symptoms and severity of the disease vary depending on the type, while the exact mechanism of many cases are remained unknown due to being in a form of rare diseases. While symptoms of muscle disease involve rapid progression, patients with muscle diseases suffer from pain that makes them difficult to live a daily life on their own with progression of the disease, but basic treatments for the related disease barely exist.

Meanwhile, obesity may be defined as a type of disease in which an excess amount of fat accumulates abnormally in the body, threatening the health of an individual. Obesity is divided into simple obesity, which is mainly caused by overeating and lack of exercise, and symptomatic obesity, which is caused by endocrine disorders. The causes of simple obesity include poor eating habits such as overeating, binge eating, snacking, late-night snacking, and irregular eating, as well as lack of exercise and side effects from medications.

With the growth of economy and changes in lifestyles, there have been many changes in eating habits in recent years. Particularly, overweight and obesity are on the rise in busy modern people due to high-calorie diets such as fast food and low amount of exercise. According to the World Health Organization (WHO), more than 1 billion adults worldwide are overweight, and at least 3 million of them are clinically obese, with reports that 250,000 people in Europe and more than 2.5 million people worldwide have died each year in association with overweight.

Being overweight and obese may increase blood pressure and cholesterol levels, which may lead to or worsen various chronic complications, including heart disease, diabetes, arthritis, fatty liver, hyperlipidemia, and cancer. In addition, overweight and obesity are known to be key factors in increasing the incidence of arteriosclerosis, hypertension, hyperlipidemia, or heart disease in children and adolescents as well as adults. Therefore, there is a growing need to recognize obesity as a disease and actively cure it.

To date, substances used for treating obesity are drugs that reduce fat absorption in the stomach or curb appetite. However, Xenical (Roche) and Alli (GlaxoSmithKline), which take effect by inhibiting lipase in the small intestine and pancreas to prevent fat absorption, have been reported to cause side effects such as gastrointestinal side effects and reduced absorption of fat-soluble vitamins. In addition, Lorcaserin (5-HT2c receptor agonist) and Qnexa (phentermine/topiramate), which show the effect of suppressing obesity through anorectic effects, may bring about side effects such as attention deficit and memory loss, there are reports that they may cause serious adverse effects such as heart valve diseases when taken together with antidepressants or migraine drugs, and sibutramine (serotonin noradrenalin reuptake inhibitor) was withdrawn in 2010 due to increased incidence of cardiovascular disease and cerebral infarction. Since obesity treatments that are currently in use have serious problems as described above, development of new obesity drugs is urgently needed.

### DISCLOSURE OF THE INVENTION

### Technical Goals

An object of the present disclosure is to provide a peptide having an amino acid sequence represented by SEQ ID NO: 1.

Another object of the present disclosure is to provide a pharmaceutical composition for treating or preventing muscle disorders, including the peptide as an active ingredient.

Another object of the present disclosure is to provide a pharmaceutical composition for preventing or treating obesity diseases, including the peptide as an active ingredient.

Another object of the present disclosure is to provide a health functional food composition for ameliorating or preventing muscle disorders, including the peptide as an active ingredient.

Another object of the present disclosure is to provide a health functional food composition for preventing or ameliorating obesity diseases, including the peptide as an active ingredient.

Another object of the present disclosure is to provide a reagent composition having an activity of promoting myoblast proliferation or myocyte differentiation, including the peptide as an active ingredient.

Another object of the present disclosure is to provide a reagent composition having an activity of suppressing preadipocyte proliferation or myocyte differentiation, including the peptide as an active ingredient.

Another object of the present disclosure is to provide a medium additive composition for myoblast culture, including the peptide as an active ingredient.

### Technical Solutions

To achieve the above object, the present disclosure provides a peptide having an amino acid sequence represented by SEQ ID NO: 1.

In addition, the present disclosure provides a pharmaceutical composition for treating or preventing muscle disorders, including the peptide as an active ingredient.

In addition, the present disclosure provides a pharmaceutical composition for preventing or treating obesity diseases, including the peptide as an active ingredient.

In addition, the present disclosure provides a health functional food composition for ameliorating or preventing muscle disorders, including the peptide as an active ingredient.

In addition, the present disclosure provides a health functional food composition for preventing or ameliorating obesity diseases, including the peptide as an active ingredient.

In addition, the present disclosure provides a reagent composition having an activity of promoting myoblast proliferation or myocyte differentiation, including the peptide as an active ingredient.

In addition, the present disclosure provides a reagent composition having an activity of suppressing preadipocyte proliferation or myocyte differentiation, including the peptide as an active ingredient.

In addition, the present disclosure provides a medium additive composition for myoblast culture, including the peptide as an active ingredient.

### Advantageous Effects

The present disclosure relates to a peptide having an amino acid sequence represented by SEQ ID NO: 1; and a pharmaceutical composition and reagent composition including the peptide as an active ingredient, wherein it was found that, after treatment of the peptide, proliferation and differentiation of myoblasts and myoregeneration increased while preadipocyte proliferation and adipodifferentiation decreased, such that the peptide may be used as a substance for 1) promotion of myoblast proliferation, 2) promotion of myoblast differentiation, 3) a substance for regeneration of damaged muscle, 3) inhibition of preadipocyte proliferation, and 4) suppression of differentiation of preadipocytes into adipocytes.

### BRIEF DESCRIPTION OF DRAWINGS

FIG. 1 shows an interaction of extracellular domains of myostatin (hereinafter, referred to as MSTN) and activin IIb receptor (ACVRIIB) in the presence and absence of a peptide (hereinafter, referred to as MIF1) having an amino acid sequence represented by SEQ ID NO: 1.

FIG. 2 shows binding amino acids of MSTN and ACVRIIB proteins in the presence or absence of MIF1.

FIG. 3 shows proliferation and differentiation of myoblasts upon treatment of MSTN proteins.

FIG. 4 shows proliferation of myoblasts upon treatment of variously modified MIF1.

FIG. 5 shows proliferation and differentiation of myoblasts upon treatment of MIF1.

FIG. 6 shows expression of Atrogin1, MuRF1, and ACVRIIB upon treatment of MIF1 during myodifferentiation.

FIG. 7 shows proliferation and differentiation of myoblasts upon treatment of _{Ac}-MIF1.

FIG. 8 shows expression of Atrogin1, MuRF1, and ACVRIIB upon treatment of _{Ac}-MIF1 during myodifferentiation.

FIG. 9 shows treatment of _{Ac}-MIF1 peptides and MSTN proteins during myodifferentiation.

FIG. 10 shows a myoregenerative effect upon treatment of _{Ac}-MIF1 peptides.

FIG. 11 shows expression of fibromodulin (hereinafter, referred to as FMOD) and myostatin (hereinafter, referred to as MSTN) according to differentiation of adipose tissues and 3T3L1 cells.

FIG. 12 shows adipodifferentiation following suppression of FMOD and MSTN expression; and relevant gene expression in adipose tissues following MSTN knock-out.

FIG. 13 shows an effect of _{Ac}-MIF1 peptide treatment in 3T3L1 cell proliferation and differentiation.

### BEST MODE FOR CARRYING OUT THE INVENTION

Hereinafter, the present disclosure will be described in detail.

The present disclosure provides a peptide having an amino acid sequence represented by SEQ ID NO: 1.

A C-terminus of the peptide may be amidated, or an N-terminus may be acetylated.

The peptide may have a myoblast proliferation or myocyte differentiation-promoting activity, and preferably, it may inhibit a myostatin (MSTN) protein to have a myoblast proliferation or myocyte differentiation-promoting activity, while suppressing proliferation of preadipocyte or suppressing differentiation into adipocytes.

In addition, the present disclosure provides a pharmaceutical composition for treating or preventing a muscle disorder, including the peptide as an active ingredient.

The muscle disorder may be, but is not limited to, one or more selected from among muscular dystrophy, muscle diseases, muscle damage, muscular dystrophy, sarcopenia, myoneural conductive diseases, or nerve damage.

In addition, the present disclosure provides a pharmaceutical composition for preventing or treating obesity diseases, including the peptide as an active ingredient.

The pharmaceutical composition may be prepared in one or more formulations selected from the group consisting of powders, granules, tablets, capsules, suspensions, emulsions, syrups, eye drops, and injection solutions.

In another example embodiment of the present disclosure, the pharmaceutical composition may further include one or more additives selected from the group consisting of appropriate carriers, excipients, disintegrators, sweeteners, coating agents, swelling agents, lubricants, glidants, flavoring agents, antioxidants, buffers, bacteriostatic agents, diluents, dispersants, surfactants, binders, and lubricants that are commonly used in the preparation of pharmaceutical compositions.

Specifically, for carriers, excipients, and diluents, lactose, dextrose, sucrose, sorbitol, mannitol, xylitol, erythritol, maltitol, starch, gum acacia, alginate, gelatin, calcium phosphate, calcium silicate, cellulose, methyl cellulose, microcrystalline cellulose, polyvinyl pyrrolidone, water, methyl hydroxybenzoate, propyl hydroxybenzoate, talc, magnesium stearate, and mineral oil may be used, solid preparations for oral administration include tablets, pills, powders, granules, and capsules, and these solid preparations may be prepared by mixing at least one or more excipients in the composition, for example, starch, calcium carbonate, sucrose or lactose, and gelatin. In addition to simple excipients, lubricants such as magnesium stearate and talc may also be used. Oral liquid preparations may include suspensions, liquid solutions, emulsions, and syrups, and may include various excipients, such as humectants, sweeteners, fragrances, and preservatives, in addition to the simple diluents that are commonly used, such as water and liquid paraffin. Preparations for parenteral administration include sterile aqueous solutions, non-aqueous solvents, suspensions, emulsions, lyophilized preparation, and suppositories. For non-aqueous solvents and suspensions, propylene glycol, polyethylene glycol, vegetable oils such as olive oil, and injectable esters such as ethyl oleate may be used. The substrates of the suppository may include witepsol, macrogol, tween 61, cacao butter, laurin butter, and glycerogelatin.

According to an example embodiment of the present disclosure, the pharmaceutical composition may be administered to a subject in an ordinary manner through the intravenous, intraarterial, intraperitoneal, intramuscular, intraarterial, intraperitoneal, intrasternal, transdermal, intranasal, inhalational, topical, rectal, oral, intraocular, or intradermal routes.

The dosage of the active ingredient according to the present disclosure may vary depending on the condition and weight of the subject, the type and severity of a disease, the drug form, the route and duration of administration and may be appropriately selected by the person skilled in the art, and the daily dose may be 0.01 mg/kg to 200 mg/kg, preferably 0.1 mg/kg to 200 mg/kg, and more preferably 0.1 mg/kg to 100 mg/kg. The administration may be conducted once a day or divided into several doses, which does not limit the scope of the present disclosure.

In addition, the present disclosure provides a health functional food composition for ameliorating or preventing muscle disorders, including the peptide as an active ingredient.

In addition, the present disclosure provides a health functional food composition for preventing or ameliorating obesity diseases, including the peptide as an active ingredient.

The health functional food may include various nutritional supplements, vitamins, minerals (electrolytes), flavor agents such as synthetic flavor agents and natural flavor agents, coloring agents and boosters (cheese, chocolate, etc.), pectic acid and salts thereof, alginate and salts thereof, organic acids, protective colloidal thickeners, pH regulators, stabilizers, preservatives, glycerin, alcohol, and carbonation agents used in carbonated beverages.

In addition, it may contain pulp for the manufacture of natural fruit juices, synthetic fruit juices, and vegetable drinks. These ingredients may be used independently or in combination. In addition, the health functional food composition may be in the form of any one of meat, sausage, bread, chocolate, candy, snacks, confectionery, pizza, noodles, chewing gum, ice cream, soups, beverages, teas, functional waters, drinks, alcohol, and vitamin complexes.

In addition, the health functional food may additionally include food additives, and its suitability as a “food additive” may be determined by the standards and criteria for the item in accordance with the general regulations of the Food Additive Code approved by the Food and Drug Administration and the general test method, unless otherwise stipulated.

The items listed in the “Korean Food Additives Codex” may include, for example, chemically synthesized compounds such as ketones, glycine, potassium citrate, nicotinic acid, and cinnamic acid, natural additives such as persimmon color, licorice extracts, crystallized cellulose, kaoliang color, and guar gum, and mixed preparations such as sodium L-glutamate preparations, noodle-added alkali agents, preservative agents, and tar color agents.

Here, the content of the active ingredient added to the food in the process of manufacturing the health functional food may be appropriately adjusted as needed, and preferably it may be added by 1 part by weight to 90 parts by weight into 100 parts by weight of the food.

In addition, the present disclosure provides a reagent composition having an activity of promoting myoblast proliferation or myocyte differentiation, including the peptide as an active ingredient.

In addition, the present disclosure provides a reagent composition having an activity of inhibiting preadipocyte proliferation or myocyte differentiation, including the peptide as an active ingredient.

In addition, the present disclosure provides a medium additive composition for myoblast culture, including the peptide as an active ingredient.

### MODES FOR CARRYING OUT THE INVENTION

Hereinafter, to help understanding of the present disclosure, examples will be described in detail. However, the following examples are merely illustrative of the contents of the present disclosure, and the scope of the present disclosure is not limited to the following examples. The examples of the present disclosure are provided to more completely explain the present disclosure to those with ordinary skill in the art.

### [Preparation Example 1] Acquisition of Peptides

MIF1, _{Ac}-MIF1, MIF1-_{NH2}, and _{Ac}-MIF 1-_{NH2} (hereinafter referred to as MIF peptides) were synthesized from Peptron, diluted with dimethyl sulfoxide (DMSO), and stored at −20° C.

### [Preparation Example 2] Culture of C2C12 Cells and Observation of Proliferation Upon MIF Peptide Treatment

C2C12 cells, a mouse myoblast line, were cultured in Dulbecco's Modified Eagle's Medium (DMEM)+10% fetal bovine serum (FBS)+1% penicillin/streptomycin (P/S). To validate the effect of MIF peptide, C2C12 cells (2×10³ cells/ml) were placed in a 12 well cell culture dish and attached for 24 hours, and MIF peptide (1000 nM) was treated for 1 day to observe cell proliferation by MTT method. The medium was replaced every 2 days, and the cells were cultured at 37° C.

### [Preparation Example 3] Mouse

The male C57BL/6 mice were purchased from Dae Han Bio Link Co., Ltd., four of which per cage were kept in a temperature-controlled room with a 12-hour light cycle. The animals were fed with a standard rodent diet containing 4.0% (wt/wt) total fat (Rodent NIH-31 Open Formula Auto; Zeigler Bros., Inc., Gardners, PA, USA) and water. All experiments involving animals complied with the guidelines (YUMC-AEC2015-006) issued by the Animal Control Committee of Youngnam University's Animal Research Institute. The MSTN knock-out mouse was provided by a laboratory at Seoul National University. Normal, MSTN+/− (heterozygous), and MSTN−/− (homozygous) adipose tissues were fixed by collecting from 6-week-old mice and stored at −80° C. until required for analysis.

### [Preparation Example 4] Culture of 3T3L1 Cells

3T3L1 cells, mouse fibroblasts, were cultured in Dulbecco's Modified Eagle's Medium (DMEM)+10% fetal bovine serum (FBS)+1% penicillin/streptomycin (P/S). To validate the effect of MIF peptide, 3T3L1 cells (2×10³ cells/ml) were placed in a 12-well cell culture dish, attached for 24 hours, and treated with MIF peptide (1000 nM) for 2 days, followed by observation of cell proliferation by MTT method. The medium was replaced every 2 days, and the cells were cultured at 37° C.

### [Experimental Example 1] Analysis on Protein-Protein Interactions

Structures of MSTN (pdb id: 3HH2) and ACVRIIB (pdb id: 1S4Y) were retrieved from the RCSB protein databank. All water molecules and hetero atoms were removed from both structures. The structure of the FMOD was modeled using a combination of initial folding and threading methods using I-TASSER (Yang Zhang Institute at the University of Michigan, Ann Arbor, USA, http://zhanglab.ccmb.med.umich). Since the low rate of sequence identity in the structure of the protein data bank of homologs may not bring out a robust model for FMOD, used was the I-TASSER server, which provides the best protein model determined by the Critical Assessment of Structure Prediction (CASP)-7 and -8, a world-class experiment designed to provide an objective assessment of state-of-the-art structures. In this analysis, all protein-protein studies were performed using PatchDock.

### [Experimental Example 2] Pattern Analysis

A series of evaluations were carried out to find out some common binding patterns. Depth analysis was conducted on the binding of MSTN for FMOD and ACVRIIB, followed by selection of the residual segments of MSTN participating most in the interaction. Pattern studies were carried out using various approaches such as changes in accessible surface area and virtual alanine scanning (available in http://robetta.bakerlab.org/alaninescan) to predict the maximum residues contributing to the complex.

### [Experimental Example 3] Peptide Screening

The efficacy of the designed peptide for MSTN was predicted using an in-silico binding approach. All peptides designed here were docked for MSTN. Combined studies were conducted using Patchdock. The results obtained from Patchdock were further enhanced using Firedock for 1000 steps each, and the top scoring peptides were selected based on the overall binding energy for MSTN. Peptide information is as shown in Table 1.

TABLE 1

| | | | Molecular | Molecular |
|---|---|---|---|---|
| Peptides | Sequence information | Size (mer) | formula | weight |
| MIF 1 | VDFEAFWDWG | 10 | C₆₃H₇₄N₁₂O₁₇ | 1270.53 |
| _{Ac}-MIF 1 | _{Ac}-VDFEAFWDWG | 10 | C₆₅H₇₆N₁₂O₁₈ | 1312.54 |

### [Experimental Example 4] Scratch Experiment

When the C2C12 cells were 100% grown, cells were scratches on the surface, treated with 1000 nM MIF1 or _{Ac}-MIF1 peptides, and cultured for 1 day, followed by observation for a degree of cell recovery.

### [Experimental Example 5] Validation of Cell Proliferation (MTT Method)

To validate cell proliferation, the culture medium of the cells was removed, the cells were washed with DMEM, 500 μl of MTT reagent (0.5 mg/ml) dissolved in phosphate buffered saline (PBS) was added to each well, and the cells were left at 37° C. for 1 hour. The reaction solution was removed, and 1000 μl of DMSO was added to each well. Purple formazan crystals were completely dissolved in DMSO, and the absorbance was measured at 540 nm.

### [Experimental Example 6] Treatment of MSTN Proteins

When C2C12 cells were 70% grown, media were replaced with myodifferentiation medium added with MSTN protein (1 ng), _{Ac}-MIF1 (1000 nM), and _{Ac}-MIF1 (1000 nM)+MSTN protein (1 ng) from proliferation medium, followed by culture for 3 days.

### [Experimental Example 7] Myodifferentiation

When the cells grew more than 70% for differentiation of myoblasts into myocytes, they were cultured for 3 days by replacing with a differentiation medium (DMEM+2% FBS+1% P/S). The medium was replaced once daily, and the cells were cultured at 37° C.

### [Experimental Example 8] Giemsa Staining and Calculation of Fusion Index

The cell medium was removed, and the cells were washed with PBS. After washing, a methanol:PBS reagent in a 1:1 volume ratio was treated, followed by fixation for 2 minutes. Additionally, after a methanol:PBS reagent in a 2:1 volume ratio was added, fixation was followed for additional 2 minutes. After 2 minutes, 0.04% Giemsa reagent was added, and then the cells were left for 30 minutes, washed with PBS after 30 minutes, and observed under a microscope, with 3 photographs taken for each cell (300×). In the photographs taken, the number of fused nuclei in the root canal cells was counted, the number of nuclei of total cells was counted, and the number of fused nuclei was divided by the number of nuclei of the total cells to obtain the % value.

### [Experimental Example 9] RNA Extraction and cDNA Synthesis

After adding 1 ml of TRIzol™ reagent, the cells were crushed using a sonicator. After centrifugation of the crushed sample (12,000 rpm, 10 min, 4° C.), the supernatant was transferred to a new tube, added with 200 μl of chloroform, and left at room temperature for 10 minutes. After 10 minutes, centrifugation (12,000 rpm, 10 min, 4° C.) was performed, yielded a transparent supernatant. Next, 500 μl of isopropanol was added, the mixture was left for 10 minutes, and centrifugation was performed to obtain RNA pellets. After washing by adding 70% ethanol (distilled water treated with ethanol+diethylpyrocarbonate (hereinafter referred to as DEPC)) to the RNA pellets, complete removal and drying were followed. The dried transparent RNA was added with DEPC-treated distilled water and then stored at −80° C. Total RNA volume was measured by nanodrop, and the 18s and 28s bands were detected in 1.2% agarose gel. cDNA was synthesized with 2 μg of total RNA, random hexamer primers, and reverse transcriptase (25° C.: 10 min, 37° C.: 120 min, 85° C.: 5 min).

### [Experimental Example 10] Detection of Gene Expression (Real-Time PCR)

Real-time PCR (RT-PCR) was performed for detecting gene expression. For observation of real-time gene expression, gene expression was analyzed using the Power SYBR Green PCR Master Mix, which includes fluorescent materials of SYBR green (7500 Real-time PCR system). The PCR primer was designed with Primer 3 software (http://frodo.wi.mit.edu) according to the nucleotide sequence secured from NCBI GenBank. PCR was performed 40 times at 95° C. for 10 minutes, again at 95° C. for 33 seconds, at gene primer temperature (tm) for 33 seconds, and at 72° C. for 33 seconds. Gene expression values were analyzed through the analysis of c(t) values obtained through real-time PCR analysis (fold change 2-^{ΔΔ}Ct formula). Setting the gene expression value of the untreated cells to 1, the gene expression value of the treated cells was calculated. In the analysis of gene c(t) values, normalization was conducted with the glyceraldehyde-3-phosphate dehydrogenase (GAPDH). The sequence of the PCR primer is as shown in Table 2.

TABLE 2

| | Size | Temper- | | |
|---|---|---|---|---|
| Primer | (bp) | ature | Forward | Reverse |
| GAPDH | 155 | 59 | 5′-tgctggt | 5′-caagcag |
| | | | gctgagtatg | ttggtggtac |
| | | | tcg-3′ | agg-3′ |
| MSTN | 163 | 59 | 5′-acgctac | 5′-ggagtct |
| | | | cacggaaaca | tgacgggtct |
| | | | atc-3′ | gag-3′ |
| MYOG | 185 | 59 | 5′-tccagta | 5′-caaatga |
| | | | cattgagcgc | tctcctgggt |
| | | | cta-3′ | tgg-3′ |
| MYL2 | 177 | 59 | 5′-aaagagg | 5′-cctctct |
| | | | ctccaggtcc | gcttgtgtgg |
| | | | aat-3′ | tca-3′ |
| MYH | 248 | 59 | 5′-gggttcc | 5′-agggcca |
| | | | attgacattg | gtgtttcaca |
| | | | acc-3′ | ttc-3′ |
| Atrogin 1 | 160 | 59 | 5′-ttcagca | 5′-tgaaagc |
| | | | gcctgaacta | ttcccccaaa |
| | | | cga-3′ | gta-3′ |
| MuRF1 | 206 | 59 | 5′-tgaggtg | 5′-tcacctg |
| | | | cctacttgct | gtggctattc |
| | | | cct-3′ | tcc-3′ |
| ACVRIIB | 197 | 59 | 5′-aacttcc | 5′-atcgtgg |
| | | | agagagacgc | gcctcatctt |
| | | | ctt-3′ | ctt-3′ |
| FMOD | 155 | 59 | 5′-tgcagaa | 5′-cttgatc |
| | | | gatccctcct | tcgttcccat |
| | | | gtc-3′ | cca-3′ |
| CD36 | 187 | 59 | 5′-tggagct | 5′-tgggttt |
| | | | gttattggtg | tgcacatcaa |
| | | | cag-3′ | aga-3′ |
| PPARγ | 232 | 59 | 5′-aagagct | 5′-acccttg |
| | | | gacccaatgg | catccttcac |
| | | | ttg-3′ | aag-3′ |
| CD163 | 128 | 59 | 5′-ctggtcg | 5′-cgccact |
| | | | tgtggaagtg | gagcatagtg |
| | | | aaa-3′ | aaa-3′ |

### [Experimental Example 11] Immunocytochemistry

The medium of cultured C2C12 cells was removed and washed once with PBS. The washed cells were treated with 4% formaldehyde (Sigma), fixed for 15 minutes, then washed using PBS, added with 0.2% trypton X-100 (Sigma), and left for 5 minutes. After washing again using PBS, adding 1% normal goat serum, and leaving for 30 minutes, the primary antibody (MYOD, myogenin (MYOG):myosin light chain (MYL2); 1:50) was added to carry out a reaction at 4° C. for 14 hours. The antibodies were removed, washing was performed 3 times with PBS for 10 minutes, and then the cells were left with secondary antibody (Alexa Fluor 488 goat anti-Mouse & rabbit SFX kit t) for 1 hour. After 1 hour, the antibodies were removed, washing was performed with PBS for 10 minutes, the nuclei were stained with 4′,6-diamidino-2-phenylindol (DAPI), and the expression of the proteins was observed using fluorescence microscopy.

### [Experimental Example 12] Western Blot

(1) The medium of cultured C2C12 cells was removed, followed by washing with PBS. After adding LIPA buffer and protease inhibitor to the washed cells, the proteins included in the supernatant were collected after centrifugation for 10 minutes at 12000 rpm. 40 μg of the extracted proteins were subjected to electrophoresis in 8 to 10% acrylamide gel and then transferred to a polyvinylidene fluoride (PVDF) membrane (Milipore). These were blocked with 3% skim milk or bovine serum albumin (BSA) at room temperature for 1 hour. Afterwards, primary antibody diluted in 1% skim milk or BSA (Pax7; 1:500, MYOD; 1:500, MYOG; 1:400, MYL2; 1:1000, MYH; 1:500, MSTN; 1:1000, β-actin; 1:1000, MuRF1; 1:400, and Atrogin; 1:1:400) was added and reacted at 4° C. for over 16 hours. After 16 hours, cells were washed three times with TBST (Tris-buffered saline with Tween 20) and reacted with secondary antibody with horseradish peroxidase (HRP) conjugated at room temperature for 1 hour. It was washed three times with TBST and developed after adding Super Signal West Pico Chemiluminescent Substrate.

(2) The medium of cultured 3T3L1 cells was removed and washed with PBS. After adding LIPA buffer and protease inhibitor to the washed cells, the proteins included in the supernatant were collected after centrifugation at 12000 rpm for 10 minutes. 40 μg of the extracted proteins were subjected to electrophoresis in 8 to 10% acrylamide gel and then transferred to a polyvinylidene fluoride (PVDF) membrane (Milipore). These were blocked with 3% skim milk or bovine serum albumin (BSA) at room temperature for 1 hour. Afterwards, primary antibody diluted in 1% skim milk or BSA (MSTN; 1:1000, 13-actin; 1:1000, CD36; 1:500, CD163; 1:500, FMOD; 1:400, and PPARγ; 1:500) was added and reacted at 4° C. for over 16 hours. After 16 hours, they were washed three times with TBST (Tris-buffered saline with Tween 20) and reacted with secondary antibody conjugated with horseradish peroxidase (HRP) at room temperature for 1 hour. It was washed three times with TBST and developed after adding Super Signal West Pico Chemiluminescent Substrate.

### [Experimental Example 13] Immunohistochemistry

Muscle tissues containing paraffin was subjected to deparaffinization with xylene and ethanol and hydration, respectively, and the tissue was soaked in 0.3% hydrogen peroxide water (H₂O₂)/methanol for 15 minutes to stop endogenous peroxidase activity. Then, tissues were stained with hematoxylin/eosin for morphological observation or reacted with 1% normal goat serum at room temperature for 1 hour to block a reaction with non-specific antibodies, followed by a reaction with the primary antibody (1:50) at 4° C. for over 16 hours. After 16 hours, washing was performed 3 times with PBS, followed by a reaction with secondary antibody (1:100) with HRP conjugated at room temperature for 1 hour. Horse radish peroxidase-conjugated streptavidin was added to detect the expression of the protein, followed by observation under a microscope.

### [Experimental Example 14] Observation of a Myoregeneration Effect Upon Injection of _{Ac}-MIF 1 Peptide

In order to observe the myoregeneration effect following MIF peptide injection, mice were injected with _{Ac}-MIF 1 peptide and injected/uninjected with cardiotoxin (hereinafter referred to as CTX) into the muscle, followed by observation of the morphology and regeneration of the muscle. A day after C57BL/6 mice were injected with _{Ac}-MIF1 peptide (1.125 mM), 100 mM cardiotoxin was injected into the gastrocnemius. Muscle tissues were sampled 7 days after CTX injection.

### [Experimental Example 15] Measurement of Muscle Diameter

Paraffin-embedded muscle sections were deparaffinized using xylene, rehydrated using a concentration gradient of ethanol, stained with hematoxylin and eosin, observed under an optical microscope, magnified at 400× scale, and photographed, followed by measurement of the diameter of muscle fibers using Image J software.

### [Experimental Example 16] Observation of Adipodifferentiation Upon MIF Peptide Treatment

For differentiation of 3T3L1 cells into adipocytes, when the cells grew about 100% or more, they were replaced with differentiation medium (DMEM+10% FBS+1% P/S+10 ug/ml insulin+1 μM dexametazone+0.5 μM IBMX (3-isobutyl-1-methylxanthine)) and cultured for 2 days. After 2 days, MIF was treated in a medium including 10 ug/ml insulin to induce cell differentiation.

### [Experimental Example 17] Oil Red O Staining

3T3L1 cells were treated with peptides and differentiated for 4 days, then the medium was removed, and the cells were washed with normal saline, treated with 10% formaldehyde, and left for 10 minutes. After 10 minutes, Oil-red O staining agents (6 (3.5 g Oil-red O reagent+1 ml 100% isopropanol):4 (distilled water)) were treated and left for 1 hour. After 1 hour, cells were washed with normal saline and then observed under a microscope. To measure the Oil-red O stained in the intracellularly differentiated cells, 100% isopropanol was added and collected, followed by measurement at 510 nm.

### [Experimental Example 18] Gene Knock-Down

3T3L1 cells were injected with FMOD, MSTN shRNA, or scrambled vector (1 ng) using transfection reagents and media according to the manufacturer's instructions. Cells were treated with puromycin (2 μg/ml), and then the cells injected with FMOD or MSTN shRNA were screened.

### [Experimental Example 19] Statistical Analysis

The significance in the difference between the mean of normalized gene expression was determined using Tukey's Studentized Range (HSD) and T tests. GAPDH was used for the internal control group, and the statistical analysis was performed by SAS ver. 9.0 program. A value of p≤0.05 was statistically significant.

### [Example 1] MIF1 Peptide Design and In-Silico Analysis

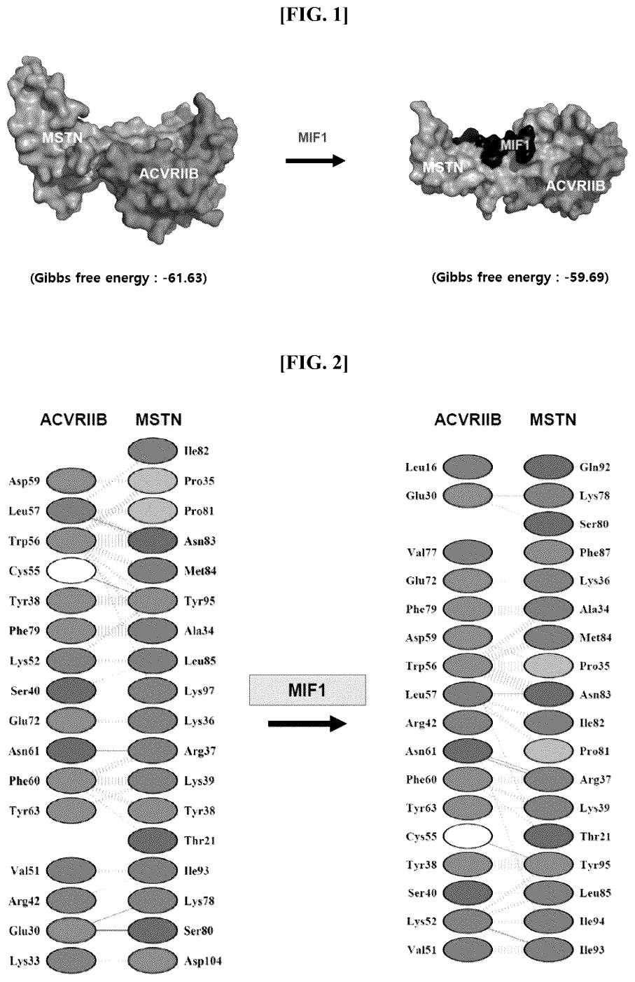

The peptide was designed at a binding site between MSTN and FMOD. As a result of analyzing the MSTN binding energy for ACVRIIB in the presence or absence of the MIF1 peptide via in-silico analysis, according to FIGS. 1 and 2, the binding energy of MSTN dropped to −59.69 in the presence of MIF1 peptide.

### [Example 2] Proliferation and Differentiation of Myoblasts Upon Treatment of MIF1

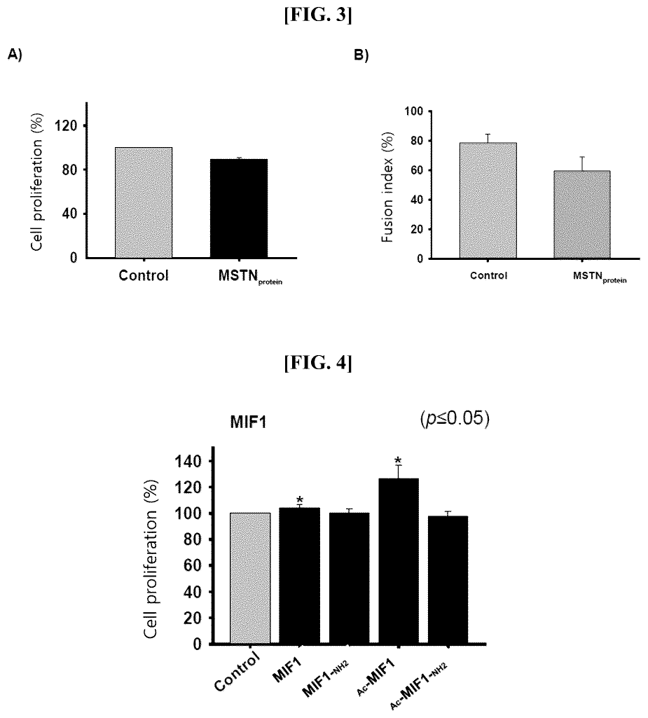

In order to observe the proliferation of cells following the treatment of MSTN protein in C2C12 cells, as a result of observing the proliferation and differentiation of cells after treatment with proliferation or differentiation media, the proliferation and differentiation of cells were reduced compared to untreated cells according to FIG. 3.

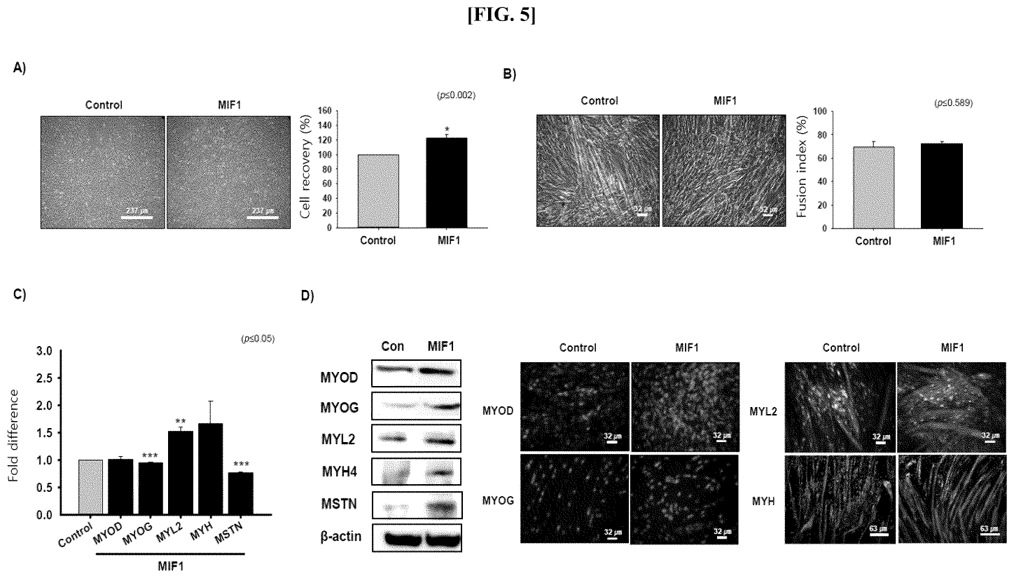

In order to block the effect of MSTN protein that inhibits the proliferation and differentiation of myoblasts and to promote the proliferation and differentiation of cells, MSTN inhibitor was carried out during the proliferation and differentiation of C2C12 cells. In order to first validate the effect by chemical modification of peptides, MSTN-inhibiting peptides (MIF1, MIF1-_{NH2}, _{Ac}-MIF 1, and _{Ac}-MIF1-_{NH2}) with various modifications were treated during the proliferation period of C2C12 cells and then observed cell proliferation with cells without peptides treatment. As a result, according to FIG. 4, cell proliferation increased in cells treated with MIF1 (11% increase compared to cells without peptide treatment) and _{Ac}-MIF1 (24% increase compared to cells without peptide treatment) compared to untreated cells (control).

MIF1 and _{Ac}-MIF1 peptides were selected for further study. To observe the effect of MIF1 peptide on proliferation of myoblasts, scratch was made on 100% grown cells which were then cultured for 1 day in proliferation medium treated with MIF1 peptide. As a result, according to FIG. 5A, the scratch recovery degree increased in cells treated with MIF1 peptide (22% increase compared to cells without peptide treatment) compared to untreated cells. After treating the cells with MIF1 peptide from the growth medium, when the cells grew more than 100%, they were treated with differentiation medium including MIF1 peptide and cultured for 3 days, followed by observation of root canal formation and fusion index. As a result, according to FIG. 5B, the fusion index of the cells increased with the treatment of the MIF1 (4% increase compared to the cells without peptide treatment) peptide compared to the untreated cells.

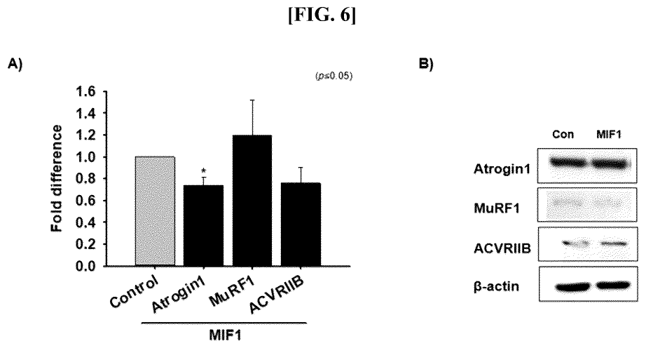

The expression of relevant myocyte differentiation-related factors was observed using real-time PCR, Western blot, and immunocytochemistry. As a result, according to C and D of FIG. 5, MYH mRNA expression increased and MSTN mRNA expression decreased in MIF1 peptide-treated cells, and expression of MYOD, MYOG, MYL2, and MYH proteins increased in MIF1 peptide-treated cells. However, expression of MSTN protein increased in cells treated with MIF1 peptides. In addition, according to FIG. 6, the expression of Atrogin1 mRNA, a factor associated with sarcopenia, decreased upon treatment of the MIF1 peptide, and the expression of the MuRF1 protein decreased.

### [Example 3] Proliferation and Differentiation of Myoblasts Upon Treatment of _{Ac}-MIF1 Peptides

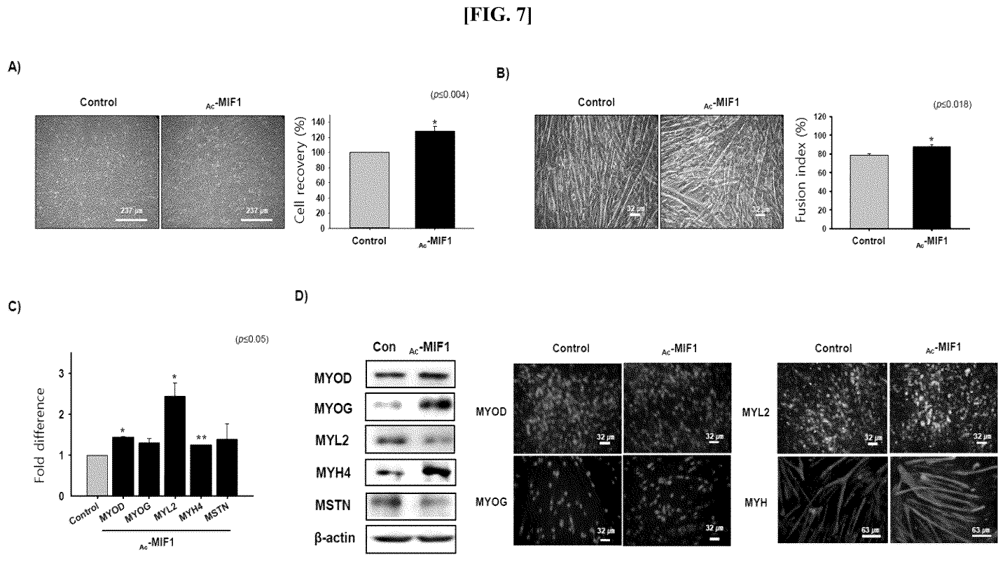

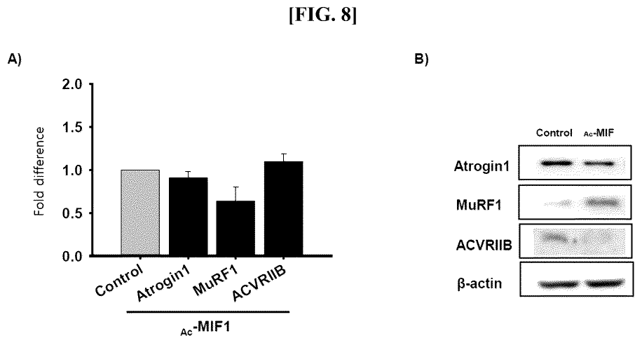

Scratch was made to observe the effect of _{Ac}-MIF1 peptide in C2C12 cells which were then cultured for 1 day with _{Ac}-MIF1-added proliferation medium, followed by measurement of cell recovery. As a result, according to FIG. 7A, the recovery of cells treated with the _{Ac}-MIF1 (28% increase compared to untreated cells) peptide was superior to that of the untreated cells.

After cells were treated with _{Ac}-MIF1 peptide from the growth medium, when the cells grew more than 100%, they were treated with differentiation medium including _{Ac}-MIF1 peptide and cultured for 3 days, followed by observation of root canal formation and fusion index. As a result, according to FIG. 7B, _{Ac}-MIF1 (an 11% increase in the fusion index with nuclei fused in cells compared to untreated cells) treatment increased root canal formation compared to untreated cells.

According to C and D in FIG. 7, expression of MYOD and MYL2 mRNA; and MYOD, MYOG, and MYH proteins increased in _{Ac}-MIF1 treated cells. However, MSTN protein expression was reduced in _{Ac}-MIF1 treated cells. In addition, according to FIG. 8, expression of Atrogin1 and ACVRIIB proteins was also reduced in _{Ac}-MIF1 peptide treatment.

### [Example 4] Effect of MSTN Protein and _{Ac}-MIF1 Peptide in Differentiation in Muscles

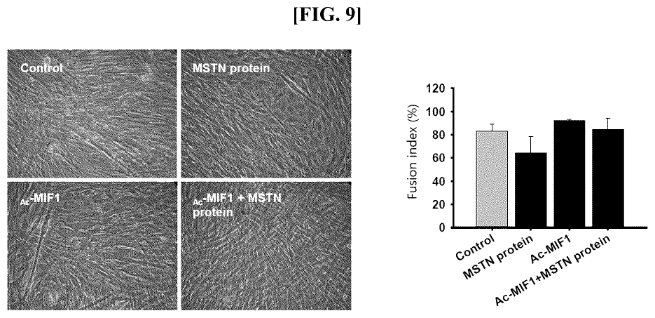

In order to observe the effect of MIF peptide treatment on MSTN protein during the differentiation of C2C12 cells, C2C12 cells were cultured up to 100% in growth medium, and, when grown 100%, they were cultured for 3 days in muscle differentiation medium including MSTN protein, _{Ac}-MIF1 or MSTN protein+_{Ac}-MIF1. The fusion index was analyzed in cells treated with MSTN protein, _{Ac}-MIF1, or MSTN protein+_{Ac}-MIF1. As a result, according to FIG. 9, root canal formation was reduced in MSTN protein-treated cells compared to untreated cells, and the fusion index of _{Ac}-MIF1 peptide-treated cells increased compared to untreated cells. Root canal formation in cells treated with MSTN protein+_{Ac}-MIF1 peptide increased compared to cells treated with MSTN protein alone.

### [Example 5] Myoregeneration Effects by Injection of _{Ac}-MIF1 Peptide

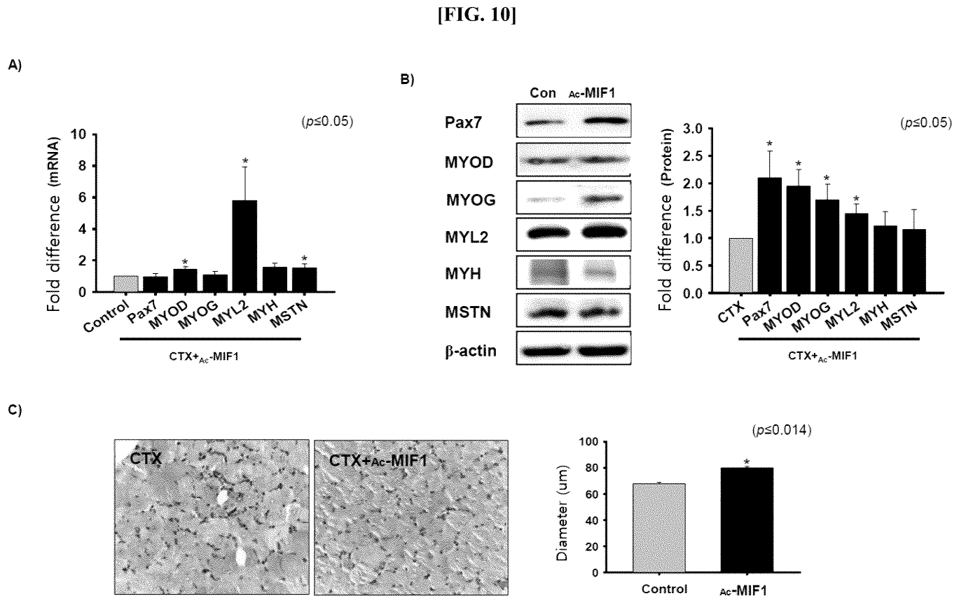

To identify the effect of _{Ac}-MIF1 peptide on the regeneration of damaged muscles, a day after _{Ac}-MIF1 was injected into the gastrocnemius of the leg, it was maintained for 7 days after CTX injection. According to Table 3, body weight (g) and muscle weight (g) were measured after 7 days, and there was no significant difference in muscle weight compared to muscle without injection in accordance with injection of _{Ac}-MIF1 peptide. According to FIG. 10, for gene expression, the mRNA expression of MYOD, MYL2, and MSTN increased in the muscle injected with _{Ac}-MIF1 peptide (FIG. 10A), the protein expression of Pax7, MYOD, MYOG, and MYL2 increased in the muscle injected with _{Ac}-MIF1 peptide (FIG. 10B), and the diameter of the muscle fibers (um) increased in the muscle injected with _{Ac}-MIF1 peptide compared to cells without peptide injection (FIG. 10C).

TABLE 3

| Peptide | CTX (g) | CTX + _{Ac}-MIF 1 (g) |
|---|---|---|
| _{Ac}-MIF 1 | 0.140 ± 0.002 | 0.140 ± 0.003 |

### [Example 6] Expression of FMOD and MSTN in Lipid

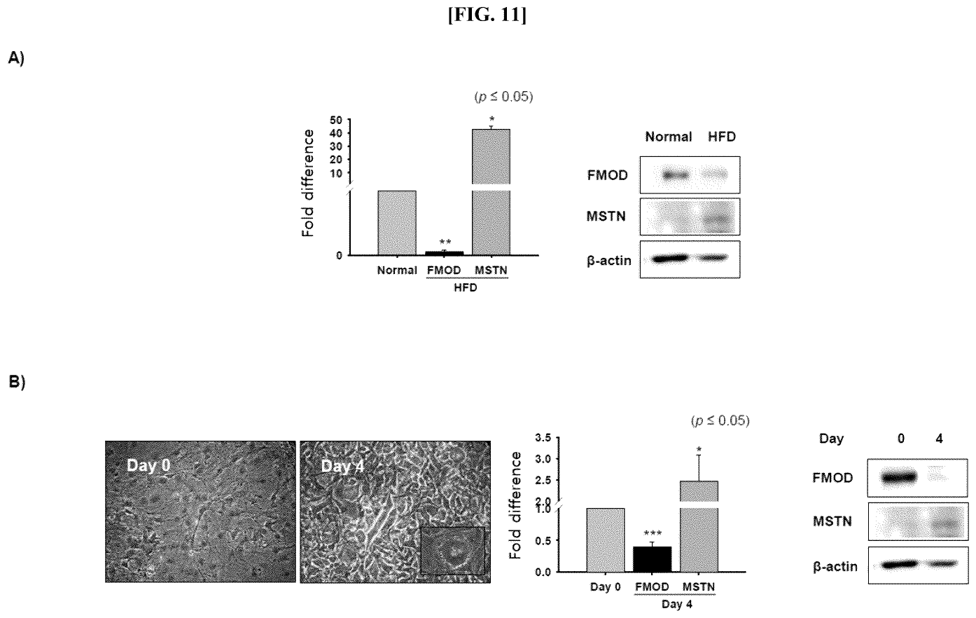

Previous studies have shown that FMOD and MSTN proteins interact to regulate MSTN expression, and inhibition of FMOD expression increases lipid accumulation in myoblasts, based on which the association between FMOD and MSTN was observed in adipose tissues or adipocytes. As a result, according to FIG. 11A, the expression of FMOD and MSTN genes was analyzed in normal and high-fat diet (HFD) mouse adipose tissues, and FMOD was reduced in HFD mouse adipose tissues, while MSTN increased. In addition, the expression of FMOD and MSTN genes was analyzed in cells before and after differentiation after subjecting the 3T3L1, mouse preadipocytes, under condition to differentiate into adipocytes for four days, and according to FIG. 11B, the expression of FMOD was reduced in differentiated adipocytes compared to pre-differentiated cells, while that of MSTN was high in differentiated adipocytes.

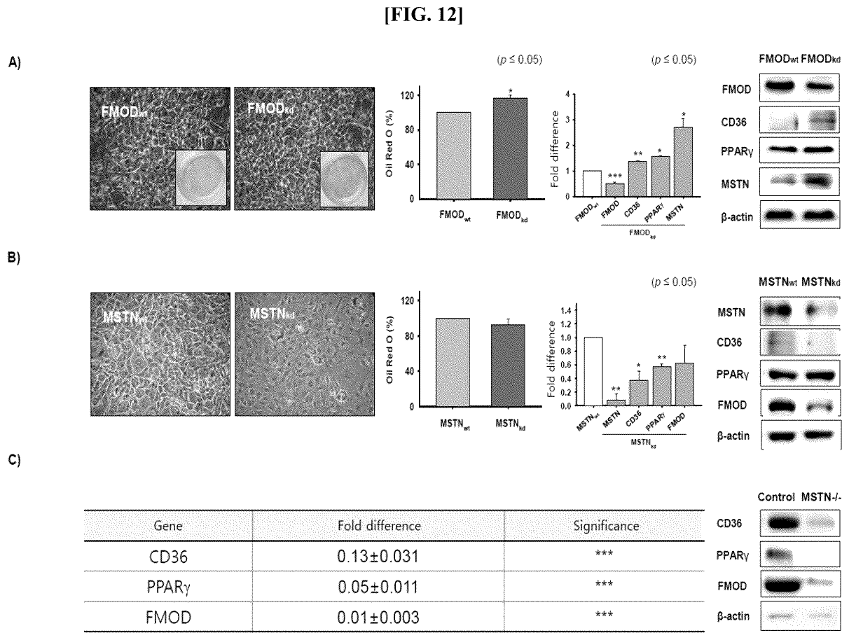

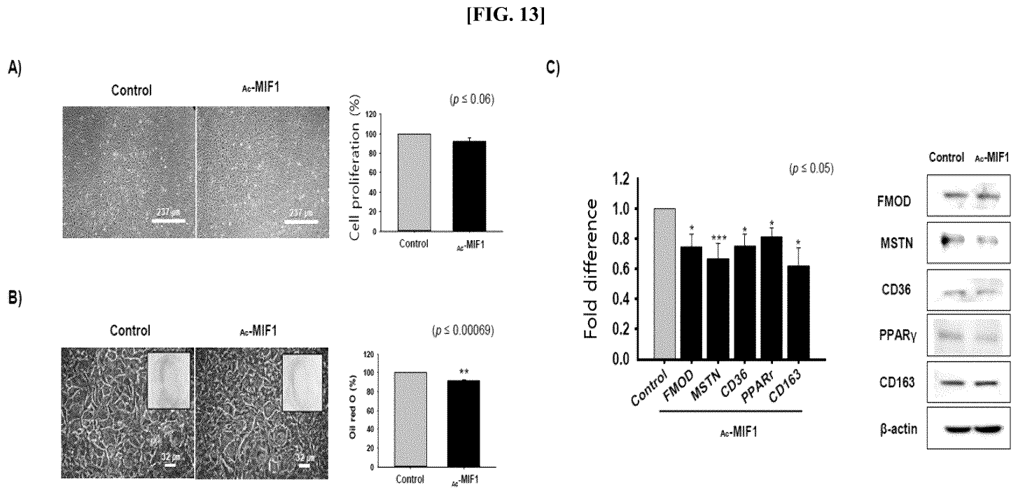

To suppress expression of FMOD or MSTN genes, after FMOD or MSTN shRNA was injected into 3T3L1 cells, adipodifferentiation was conducted, and the expression of adipodifferentiation-related factors was identified. As a result, according to FIG. 12, expression of CD36, PPARγ, and MSTN increased in cells where FMOD expression was suppressed while expression of CD36, PPARγ, and FMOD decreased in cells where MSTN expression was suppressed (A and B in FIG. 12), and expression of CD36, PPARγ, and FMOD in MSTN knock-out muscle significantly decreased compared to normal muscle tissues (FIG. 12C). Based on these data, MIF peptides derived from FMOD and MSTN binding sites were treated in the process of proliferation and differentiation of 3T3L1 cells.

### [Example 7] Proliferation and Differentiation of 3T3L1 Cells Upon Treatment of _{Ac}-MIF1

As a result of culturing 3T3L1 cells for 2 days in a proliferation medium added with _{Ac}-MIF1 and then measuring the proliferation, according to FIG. 13A, it decreased in _{Ac}-MIF1 peptide treated cells (8% increase compared to cells without peptide treatment) compared to untreated cells.

When 3T3L1 cells reached 100% growth, they were treated with adipocyte-induced differentiation medium added with _{Ac}-MIF 1 peptide and cultured for 4 days. As a result, according to FIG. 13B, adipodifferentiation was observed by Oil-red O staining, Oil-red O intensity was measured in _{Ac}-MIF1 treated and untreated cells, and fat accumulation was reduced in cells treated with _{Ac}-MIF1 (8% decrease compared to cells without peptide treatment) peptides.

Also, according to FIG. 13C, expression of FMOD; MSTN; and lipogenesis-related mRNAs and proteins (CD36, CD163, and PPARγ) decreased in cells treated with _{Ac}-MIF1 peptides compared to untreated cells.

The foregoing description of the present disclosure is for illustrative purposes only, and a person of ordinary skill in the art to which the disclosure pertains will understand that it may easily be modified into other concrete forms without altering the technical idea or essential features of the present disclosure. Therefore, the example embodiments described above should be understood in all respects as illustrative and not restrictive.

The scope of the present disclosure is indicated by the claims described below, and all changes or modified forms derived from the meaning and scope of the claims and their equivalent concepts should be construed as being included in the scope of the present disclosure.

1. A peptide having an amino acid sequence represented by SEQ ID NO: 1.

2. The peptide of claim 1, wherein a C-terminus of the peptide is amidated, or an N-terminus is acetylated.

3. The peptide of claim 1, wherein the peptide has a myoblast proliferation or myocyte differentiation-promoting activity; or inhibits a myostatin (MSTN) protein to suppress proliferation of preadipocytes or suppress differentiation into adipocytes.

4. The peptide of claim 3, wherein the peptide inhibits the myostatin (MSTN) protein to have a myoblast proliferation or myocyte differentiation-promoting activity.

5. A method of treating or preventing a muscle disorder, comprising:
administering a pharmaceutical composition comprising a peptide having an amino acid sequence represented by SEQ ID NO: 1 as an active ingredient to a subject.

6. The method of claim 5, wherein the muscle disorder is one or more selected from among muscular dystrophy, muscle diseases, muscle damage, muscular dystrophy, sarcopenia, myoneural conductive diseases, or nerve damage.

7. The method of claim 5, wherein the method is for preventing or treating obesity diseases.

8. The method of claim 5, wherein the pharmaceutical composition is prepared in one or more formulations selected from the group consisting of powders, granules, tablets, capsules, suspensions, emulsions, syrups, eye drops, and injection solutions.

9. A method of ameliorating or preventing a muscle disorder, comprising:
administering a health functional food composition comprising a peptide having an amino acid sequence represented by SEQ ID NO: 1 as an active ingredient to a subject.

10. The method of claim 9, wherein the method is for preventing or ameliorating obesity diseases.

11. The peptide of claim 1, wherein the peptide is included as an active ingredient in a reagent composition having an activity of promoting myoblast proliferation or myocyte differentiation.

12. The peptide of claim 1, wherein the peptide is included as an active ingredient in a reagent composition having an activity of suppressing preadipocyte proliferation or myocyte differentiation.

13. The peptide of claim 1, wherein the peptide is included as an active ingredient in a medium additive composition for myoblast culture.
